(19) United States
(12) Patent Application Publication
(10) Pub. No.: US 2005/0244351 A1
(43) Pub. Date: Nov. 3, 2005

(54) Cosmetic compositions with interpenetrating polymer network

(76) Inventors: Gale McElroy Reinhart, Middletown, NJ (US); Julio Gans Russ, Westfield, NJ (US); Tatyana Zamyatin, Princeton Junction, NJ (US); Kristen Allison Lattner, Middlesex, NJ (US)

Correspondence Address: Julie Blackburn;Revlon Consumer Products Corporation, 237 Park Avenue, New York, NY, 10017, US

(21) Appl. No.: 10/837,948

(22) Filed: May 3, 2004

(51) Int. Cl. A61K 7/021; A61K 7/025; A61K 7/32; A61K 7/06; A61K 7/11

(52) U.S. Cl. 424/63; 424/64; 424/70.12

(57) ABSTRACT

A cosmetic composition comprising an interpenetrating polymer network in a cosmetically acceptable carrier, a method for improving the transfer resistance and long wearing properties of cosmetic compositions; and a method for reducing the shiny appearance of skin or otherwise improving skin imperfections.

### TECHNICAL FIELD

The invention is in the field of cosmetic compositions for application to keratinous surfaces such as skin, nails, or hair, for the purpose of coloring, conditioning, treating, or beautifying the keratinous surface.

### BACKGROUND OF THE INVENTION

The use of silicone elastomers in cosmetic compositions is well known. Typically, silicone elastomers are crosslinked silicones generally formed by the reaction of a dimethyl methylhydrogen siloxane and a second molecule having at least some unsaturated carbon atoms, such as alpha omega dienes, silicone chains with terminal olefinic unsaturation, and the like. The crosslinking reaction generally takes place in the presence of a platinum catalyst. More recently, derivative silicone elastomers have been commercialized. Examples of such derivatives include those with EO/PO (ethylene oxide/propylene oxide) groups, which cause the elastomer to exhibit certain surfactant properties. Due to their crosslinked structure, silicone elastomers form a network, and when combined with silicone oils and other similar liquid ingredients the elastomer will often gel the silicone oils. In addition, cosmetics formulated with silicone elastomers have excellent slip and aesthetics, which is why they are so popular.

One problem with typical silicone elastomers is that they do not always exhibit good compatibility with organic or hydrocarbon (e.g. non-silicone) oils. In addition, certain silicone elastomers may not absorb oil to the degree desired for use in cosmetic formulas. In some cases, the oil absorbed into the elastomer network exudes from the network when other cosmetic ingredients are added, or the network itself doesn't absorb the amount of oil desired.

It has been discovered that a certain non-elastomeric silicone ingredient, referred to as an interpenetrating (or interpenetration) polymer network of non-covalently bonded polydimethylsiloxane and polymethylsilsesquioxane, when used in cosmetic compositions, provides certain desirable properties of elastomers, as well as improved compatibility with cosmetic oils, higher oil absorption capacity, and other beneficial properties.

It is an object of the invention to provide cosmetic compositions with excellent feel and slip.

It is a further object of the invention to provide cosmetic compositions having excellent transfer resistance or long wearing property.

It is a further object of the invention to provide cosmetic compositions which reduce the shiny appearance of skin or otherwise improve the appearance of skin imperfections.

It is a further object of the invention to provide cosmetic compositions containing an interpenetrating polymer network of at least two non-covalently bonded silicones.

### BACKGROUND OF THE INVENTION

The invention comprises a cosmetic composition comprising an interpenetrating polymer network comprised of at least one polydimethylsiloxane and at least one polymethysilsesquioxane in a cosmetically acceptable carrier.

The invention further comprises a color cosmetic composition containing an interpenetrating polymer network comprised of at least one polydimethylsiloxane and at least one polymethylsilsesquioxane in cosmetically acceptable carrier comprising at least one colorant.

The invention further comprises a method for increasing the transfer resistance or long wearing property of a cosmetic composition comprising formulating said composition with at least one interpenetrating polymer network.

The invention is further directed to a method for reducing the shiny appearance of skin or otherwise improving skin imperfections comprising applying to the skin a cosmetic composition comprising at least one interpenetrating polymer network in a cosmetically acceptable carrier.

### DETAILED DESCRIPTION

The cosmetic compositions of the invention contain at least one interpenetrating polymer network comprised of at least one polydimethylsiloxane and at least one polymethylsilsesquioxane in a cosmetically acceptable carrier that may be anhydrous or in the water and oil emulsion form. The interpenetrating polymer network and cosmetically acceptable carrier will be further described herein.

I. The Intepenetrating Polymer Network

The interpenetrating polymer network used in the compositions of the invention comprises at least two interlacing polymer networks of polydimethylsiloxane and polymethylsilsesquioxane. While the at least two silicone polymers interlace with each other on a molecular level, they are not covalently bonded. This type of polymeric structure is referred to as an “Interpenetrating Polymer Network” or “IPN”, and is something more than just a mixture of two polymeric materials. Both the physical and chemical properties of each individual polymer are changed, and the result of the combination is a new material with unique properties. Preferably the IPN is an off-white powder which may be dispersed or solubilized in volatile or nonvolatile silicones, preferably low viscosity silicones such as cyclomethicone (D4, D5, D6), linear volatile dimethicone (such as hexamethyldisiloxane (“LD2”) octamethyltrisiloxane (“LD3”), decamethyltetrasiloxane (“LD4”), dodecamethylpentasiloxane (“LD5”), and LD6) where L means linear, and D means the degree of polymerization, and the numeral following D means the number of repeating Si—O units in the polymer.

Preferably, the cosmetic compositions of the invention comprise from about 0.001-85%, preferably 0.005-80%, more preferably 0.01-75% by weight of the total composition. One type of IPN that may be used in the compositions of the invention may be purchased from Grant Industries, Inc., in Elmwood, N.J., under the tradename Gransil EPSQ, with the INCI name for such material applied for and still pending. Another type of IPN that may be used in the compositions of the invention is made by Active Concepts in South Plainfield N.J. and is sold under the trade name SilDerm Powder EPSQ, having the tentative INCI name polydimethylsiloxane/polymethylsilsesquioxane copolymer.

II. Other Ingredients

The IPN may be incorporated into a wide variety of cosmetic compositions for skin, nails, or hair, such as lotions, creams, gels, shampoos, hair conditioners, sprays, mousses; color cosmetics such as mascara, eyeshadow, blush, eyeliner, brow color, foundation, concealer, lipstick, lipliner, and so on. The compositions may be in the anhydrous or water and oil emulsion form. If the latter, the emulsions may be water-in-oil or oil-in-water. Generally emulsions comprise from about 0.1-95%, preferably about 0.1-85%, more preferably about 1-75% water and about 0.1-95%, preferably about 0.1-85%, more preferably about 1-75% oil. Examples of such other ingredients include, but are not limited to, those set forth herein.

A. Oils

Whether in the emulsion or anhydrous form, the compositions of the invention may comprise one or more oils. The term “oil” in the context of this invention means an animal, vegetable, mineral, synthetic, or silicone oil that is liquid or semi-solid at room temperature. The oil may be volatile or non-volatile. The term “volatile” means that the oil has a vapor pressure of greater than about 2 mm. of mercury at 20° C. The term “non-volatile” means that the oil has a vapor pressure of less than about 2 mm. of mercury at 20° C. If present, suggested ranges of oil found in the compositions of the invention are from about 0.1-80%, preferably about 0.5-75%, more preferably about 1-70% by weight of the total composition. Examples of oils suitable for use in the composition include, but are not limited to those set forth herein.

1. Silicone Oils

Suitable silicone oils include volatile linear or cyclic silicones. Generally such silicones have a viscosity ranging from about 0.1 to 10 centistokes at 25° C. If present, suggested ranges of volatile silicone are from about 0.1-80%, preferably about 0.5-75%, more preferably about 1-65% by weight of the total composition.

Cyclic silicones (or cyclomethicones) are of the general formula: 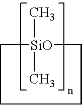
where n=3-6.

Linear volatile silicones that may be used in the compositions of the invention generally having the formula:
(CH₃)₃Si—O—[Si(CH₃)—₂—O]ₙ—Si(CH₃)₃
where n=0-7, preferably 0-5, more preferably 1-4. Examples of such linear volatile silicones include hexamethyldisiloxane, octamethyltrisiloxane, decamethyltetrasiloxane, dodecamethylpentasiloxane, and mixtures thereof.

Linear and cyclic volatile silicones are available from various commercial sources including Dow Corning Corporation and General Electric. The Dow Coming volatile silicones are sold under the tradenames Dow Coming 244, 245, 344, and 200 fluids. These fluids comprise octamethylcyclotetrasiloxane, decamethylcyclopentasiloxane, cyclohexasiloxane, and mixtures thereof.

Also suitable for use in the compositions of the invention are various non-volatile silicone oils, both water soluble and water insoluble. Such silicones preferably have a viscosity ranging from about 5 to 1,000,000 centipoise, preferably 10 to 700,000 centipoise at 25° C. Suitable water insoluble silicones include amine functional silicones such as amodimethicone; phenyl substituted silicones such as phenyl trimethicone, phenyl dimethicone, dimethicone, diphenyl dimethicone, dimethiconol, C₁₋₃₀ alkyl substituted dimethicones such as cetyl dimethicone, isostearyl dimethicone, and the like. These types of silicones are available from a variety of sources including Dow Coming Corporation, G E Silicones, Wacker, and the like.

Water soluble or dispersible silicones such as dimethicone copolyol, dimethiconol, or alkyl dimethicone copolyols (for example cetyl dimethicone copolyol), and similar, may also be used. Such silicones are available from Dow Corning as the 3225C formulation aid, Dow 190 and 193 fluids, or similar products marketed by Goldschmidt under the ABIL tradename.

2. Hydrocarbons

The oil may comprise one or more volatile or non-volatile hydrocarbon oils. Examples of volatile hydrocabons include various straight or branched chain paraffinic hydrocarbons having 5 to 26 carbon atoms, more preferably 8-16 carbon atoms. Suitable hydrocarbons include pentane, hexane, heptane, octane, decane, dodecane, tetradecane, tridecane, and C₈₋₂₀ isoparaffins such as isododecane, isohexadecane, and those disclosed in U.S. Pat. Nos. 3,439,088 and 3,818,105, both of which are hereby incorporated by reference. Preferred volatile paraffinic hydrocarbons have a molecular weight of about 70-225, preferably about 160 to 190 and a boiling point range of 30 to 320°, preferably 60-260° C., and a viscosity of less than about 10 centipoise at 25° C. Such paraffinic hydrocarbons are available from EXXON under the ISOPARS trademark, and from the Permethyl Corporation.

Suitable nonvolatile hydrocarbon oils include longer chain isoparaffins and olefins, preferably those having greater than about 18 to 20 carbon atoms. Examples of such hydrocarbon oils include C₂₄₋₂₈ olefins, C₃₀₋₄₅ olefins, C₂₀₋₄₀ isoparaffins; polyisobutene, polydecene, polybutene, and hydrogenated derivatives thereof; mineral oil, pentahydrosqualene, squalene, squalane, and mixtures thereof.

Also suitable are lower organic liquids including saturated or unsaturated, substituted or unsubstituted branched or linear or cyclic organic compounds that are liquid under ambient conditions. Preferred organic liquids include those described in U.S. Pat. Nos. 5,505,937; 5,725,845; 5,019,375; and 6,214,329, all of which are incorporated by reference herein in their entirety. Such silicones or organic oils include those further described as follows:

3. Esters

Suitable esters that may be used in the compositions of the invention are mono-, di-, and triesters. The composition may comprise one or more esters selected from the group, or mixtures thereof.

(a). Monoesters

Monoesters are defined as esters formed by the reaction of a monocarboxylic acid having the formula R—COOH, wherein R is a straight or branched chain saturated or unsaturated alkyl having 2 to 50 carbon atoms, or phenyl; and an alcohol having the formula R-OH wherein R is a straight or branched chain saturated or unsaturated alkyl having 2-50 carbon atoms, or phenyl. Both the alcohol and the acid may be substituted with one or more hydroxyl groups, or may contain other groups such as ester, ether, and the like. Either one or both of the acid or alcohol may be a “fatty” acid or alcohol, and may have from about 6 to 30 carbon atoms. Examples of monoester oils that may be used in the compositions of the invention include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, cetyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

(b). Diesters

Suitable diesters that may be used in the compositions of the invention are formed by the reaction of a dicarboxylic acid and an aliphatic or aromatic alcohol, or the reaction of an aliphatic or aromatic alcohol having at least two hydroxyl groups with one or more carboxylic acids. The dicarboxylic acid may contain from 2 to 50 carbon atoms, and may be in the straight or branched chain, saturated or unsaturated form. The dicarboxylic-acid may be subsituted with one or more hydroxyl groups. The aliphatic or aromatic alcohol may also contain 2 to 50 carbon atoms, and may be in the straight or branched chain, saturated, or unsaturated form. The aliphatic or aromatic alcohol may be substituted with one or more substitutents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, esters of neopentyl glycol such as neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.

(c). Triesters

Suitable triesters comprise the reaction product of a tricarboxylic acid and an aliphatic or aromatic alcohol, or alternatively, the reaction of an aliphatic or aromatic alcohol having at least three hydroxyl groups with one or more carboxylic acids. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate, tridecyl cocoate, tridecyl isononanoate, and so on.

(d). Tetraesters

Suitable tetraesters comprise the reaction product of alcohols having four hydroxyl groups such as pentaerythritol, with carboxylic acids which may be the same or different, and as described above with respect to the mono-, di-, and triesters. Examples of such tetraesters include esters of pentaerythritol and C₁₋₃₀ monocarboxylic acids. All of the hydroxyl groups may be reacted with monocarboxylic acids, or only one, two, or three.

(e). Lanolin Oil

Also suitable for use in the composition is lanolin oil or derivatives thereof containing hydroxyl, alkyl, or acetyl groups, such as hydroxylated lanolin, isobutylated lanolin oil, acetylated lanolin, acetylated lanolin alcohol, and so on.

(f). Glyceryl Esters of Fatty Acids

The nonvolatile oil may also comprise naturally occuring or synthetic glyceryl esters of fatty acids, or triglycerides. Both vegetable and animal sources may be used. Examples of such oils include castor oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable are synthetic or semi-synthetic glyceryl esters, e.g. fatty acid mono-, di-, and triglycerides which are natural fats or oils that have been modified, for example, acetylated castor oil, or mono-, di- or triesters of polyols such as glyceryl stearate, diglyceryl diiosostearate, polyglyceryl-4 isostearate, polyglyceryl-6 ricinoleate, glyceryl dioleate, glyceryl diisotearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(g). Fluorinated Oils

Also suitable as for use in the composition are various fluorinated oils such as fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference.

Perfluoropolyethers like those disclosed in U.S. Pat. Nos. 5,183,589, 4,803,067, 5,183,588 all of which are hereby incorporated by reference, which are commercially available from Montefluos under the trademark Fomblin.

Fluoroguerbet esters are also suitable oils. The term “guerbet ester” means an ester that is formed by the reaction of a guerbet alcohol having the general formula: 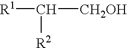
and a fluoroalcohol having the following general formula:
CF₃-(CF₂)ₙ-CH₂—CH₂—OH
wherein n is from 3 to 40.
with a carboxylic acid having the general formula:
R³COOH, or
HOOC—R³—COOH
wherein R¹, R², and R³ are each independently a straight or branched chain alkyl.

The guerbet ester may be a fluoro-guerbet ester, which is formed by the reaction of a guerbet alcohol and carboxylic acid (as defined above), and a fluoroalcohol having the following general formula:
CF₃-(CF₂)ₙ-CH₂—CH₂—OH
wherein n is from 3 to 40.

Examples of suitable fluoro guerbet esters are set forth in U.S. Pat. No. 5,488,121, which is hereby incorporated by reference. Suitable fluoro-guerbet esters are also set forth in U.S. Pat. No. 5,312,968, which is hereby incorporated by reference.

B. Natural or Synthetic Waxes

A variety of waxes may be used in the compositions of the invention including animal, vegetable, mineral, or silicone waxes. If present in the composition, the waxes may range from about 0.1-50%, preferably about 0.5-40%, more preferably about 1-38% by weight of the total composition. Generally such waxes have a melting point ranging from about 28 to 125° C., preferably about 30 to 100° C. Examples of animal, vegetable, or mineral waxes include acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax.

Also suitable are various types of ethylene homo- or copolymeric waxes such as polyethylene (also referred to as synthetic wax), polypropylene, and mixtures thereof.

Also suitable are various types of silicone waxes, referred to as alkyl silicones, which are polymers that comprise repeating dimethylsiloxy units in combination with one or more methyl-long chain (C₁₆₋₃₀) alkyl units where the long chain alkyl is preferably a fatty chain that provides a wax-like characteristic to the silicone. Such silicones include, but are not limited to stearoxydimethicone, behenoxy dimethicone, stearyl dimethicone, cetearyl dimethicone, cetyl dimethicone, and so on. Suitable waxes are set forth in U.S. Pat. No. 5,725,845, which is hereby incorporated by reference in its entirety.

C. Rheological Additives

The compositions of the invention may comprise one or more rheological additives. The term “rheological additive” means an ingredient or combination of ingredients that increase the viscosity of, or thicken, the composition, and if particulates are present, may also suspend the particulates in the composition. If a rheological additive is present, most desired is one that is a non-matting rheological additive, which means that it exhibits a reduced tendency to mute or matte the shininess of the IPN. Suggested ranges of rheological additive are from about 0.01-60%, preferably about 0.05-50%, more preferably about 0.1-45% by weight of the total composition.

One type of non-matting rheological additive comprises natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, bentones, and the like. Another example of such a rheological additive is silicate metal silicate gelling agents, such as those sold under the tradename Laponite®.

Also suitable as rheological additives are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonyphenyl

Another type of rheological additive that may be used in the compositions are silicas, silicates, silica silylate, and derivatives thereof. These silicas and silicates are generally found in the particulate form.

Also suitable as rheological additives are cross-linked organosiloxane compounds also known as silicone elastomers. Such elastomers are generally prepared by reacting a dimethyl methylhydrogen siloxane with a crosslinking group comprised of a siloxane having an alkylene group having terminal olefinic unsaturation or with an organic group having an alpha or omega diene. Examples of suitable silicone elastomers for use as rheological additives include Dow Coming 9040, sold by Dow Coming, and various elastomeric silicones sold by Shin-Etsu under the KSG tradename including KSG 15, KSG 16, KSG 19 and so on.

D. Plasticizers

It may also be desirable to include plasticizers in the compositions of the invention. Plasticizers may improve the spreadability and application of the composition to the surface to which it is applied and in some cases will interact with the film forming polymer to make it more flexible. If present, the plasticizer may be found in the oil or water phase if the composition of the invention is in the form of an emulsion, and in the oil or lipophilic phase if the composition is in the anhydrous form. A variety of plasticizers are suitable including glyceryl, glycol, and citrate esters as disclosed in U.S. Pat. No. 5,066,484, which is hereby incorporated by reference. Examples of such esters include glyceryl tribenzoate, glyceryl triacetate, acetyl tributyl citrate, dipropylene glycol dibenzoate, and the like. Also suitable, are plasticizers of the following general formula: 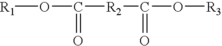
wherein R₁, R₂, and R₃ are each independently a C₁₋₂₀ straight or branched chain alkyl or alkylene which may be substituted with one or more hydroxyl groups. Preferably, R₁ is a C₃₋₁₀ straight or branched chain alkyl; R₂ is a C₂₋₈ alkyl which may be substituted with one or more hydroxyl groups; and R₃ is a C₃₋₁₀ straight or branched chain alkyl. Examples of such compounds include dioctyl malate, diisopropyl adipate, dibutyl adipate, dibutyl sebacate, dioactyl azelate, dioctyl succinate, dioctyl fumarate, and the like.

E. Particulates

The composition of the invention may contain one or more of a variety of particulates such as pigments, powders, or mixtures thereof. The particulate may be colored or non-colored (for example, white), and may have a particle size ranging from about 0.005 microns to 200 microns, preferably about 0.005 microns to 500 microns. If present, the particulate is preferably present at ranges from about 0.001-95%, preferably about 0.005-90%, more preferably about 0.01-70% by weight of the total composition. Suitable particulates include organic and inorganic pigments, powders, or salts thereof.

Examples of suitable organic pigments include red, green, blue, yellow, violet, orange, and mixtures thereof. Also suitable are Lakes of such pigments, which means that the organic pigments are reacted with a metal salt such as calcium, aluminum, barium, zirconium, and the like to form salts. Particularly preferred are aluminum Lakes of the organic pigments, which is where the organic pigment is reacted with aluminum to form the aluminum salt. Formation of the metal salt of the organic pigment will generally also convert the pigment from a water soluble pigment into a water insoluble pigment, and such pigments tend to become even more lipophilic in nature, meaning that they will exhibit appreciable affinity for lipophilic or oily ingredients in the composition. Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof. Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors that are approved for use in foods, drugs, and cosmetics by the FDA. Certified D&C and FD&C colors are listed in 21 CFR 74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 CFR 82.51. Particularly preferred are Lakes formed by the reaction of the organic pigment with a metallic salt such as aluminum, calcium, zirconium, barium, and the like. Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40. Also suitable are Lakes of such red pigments. Typically the metal salts are aluminum, barium, and the like. Most preferred are Aluminum Lakes of the various red pigments mentioned herein.

Suitable yellows include wherein the yellow pigment is a pyrazole, monoazo, fluoran, xanthene, quinoline, or salt thereof. Suitable yellows include Yellow 5, 6, 7, 8, 10, and 11, as well as Lakes of such yellow pigments.

Suitable violets include those from the anthroquinone family, such as Violet 2 and Lakes thereof. Examples of orange pigments are Orange 4, 5, 10, 11, or Lakes thereof.

Also suitable are inorganic pigments that include iron oxides such as red, blue, black, green, and yellow; titanium dioxide, bismuth oxychloride, and the like. Preferred are iron oxides.

Examples of particulates that are filler-type materials that may be included in the composition include non-pigmentitious particles that generally have a particle size ranging from about 0.002 to 200, preferably 0.5 to 100, microns. Suitable particulate fillers include titanated mica, fumed silica, spherical silica, polymethylmethacrylate, micronized teflon, boron nitride, mica, acrylates copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silk powder, silica, talc, mica, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof. The particulates may also be in the fiber form, such as cellulose fibers, rayon fibers, nylon or silk fibers and the like. Such fibers are generally circular in cross-section and have a discernable length. Preferably the length ranges from 1 to 5 mm.

The above mentioned pigments, powders or fibers may be inherently lipophilic, meaning that they are capable, alone, of being dispersed or solubilized in the oily or lipophilic phase of a cosmetic composition. The above mentioned pigments, powders, or fibers may also be inherently hydrophilic, meaning that they are capable, alone, of being dispersed or solubilized in the water phase or hydrophilic phase of a cosmetic composition. For example, certain types of organic pigments may be hydrophilic in nature and will be soluble or dispersible in water, however these pigments may, at the same time be dispersible in an oily phase. Further, in the case of organic pigments, such pigments may be reacted with metal salts to form Lakes, which cause the pigments to exhibit a more lipophilic character. In the compositions of the invention, the pigments may be inherently lipophilic or hydrophilic by virtue of surface treatments with lecithin, amino acids, mineral oil, silicone oil or various other agents either alone or in combination, which coat the particulate surface and render the particles more lipophilic in nature. The term “lipophilic” means that the pigment or particles will be solvated, dispersed, and/or compatible with the lipophilic or oily phase of the cosmetic composition. In the case of an emulsion, a lipophilic particle will have an affinity for the oily phase of the emulsion.

F. Surfactants

The compositions of the invention may comprise about 0.01-20%, preferably about 0. 1-15%, more preferably about 0.5-10% by weight of the total composition of a surfactant. Surfactants may be used in both anhydrous and emulsion based compositions. The surfactant may be nonionic, although if the composition is in the form of a shampoo or conditioner it will preferably contain anionic or cationic surfactants, respectively.

Suitable nonionic surfactants or emulsifiers include alkoxylated alcohols, or ethers, formed by the reaction of an alcohol with an alkylene oxide, usually ethylene or propylene oxide. Preferably the alcohol is a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Preferred are alkoxylated alcohols which are formed by the reaction of stearic acid, methyl glucose, and and ethoxylated alcohol, otherwise known as PEG-20 methyl glucose sesquiisostearate.

Also suitable as the nonionic surfactant are alkyoxylated carboxylic acids, which are formed by the reaction of a carboxylic acid with an alkylene oxide or with a polymeric ether. The resulting products have the general formula: 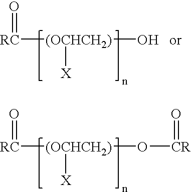
where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

Also suitable as nonionic surfactants are monomeric, homopolymeric and block copolymeric ethers. Such ethers are formed by the polymerization of monomeric alkylene oxides, generally ethylene or propylene oxide. Such polymeric ethers have the following general formula: 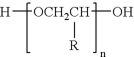
wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

Suitable cationic, anionic, zwitterionic, and amphoteric surfactants are disclosed in U.S. Pat. No. 5,534,265, which is hereby incorporated by reference in its entirety.

G. Sunscreens

If desired, the compositions of the invention may contain 0.001-20%, preferably 0.01-10%, more preferably 0.05-8% of one or more sunscreens. A sunscreen is defined as an ingredient that absorbs at least 85 percent of the light in the UV range at wavelengths from 290 to 320 nanometers, but transmits UV light at wavelengths longer than 320 nanometers. Sunscreens generally -work in one of two ways. Particulate materials, such as zinc oxide or titanium dioxide, as mentioned above, physically block ultraviolet radiation. Chemical sunscreens, on the other hand, operate by chemically reacting upon exposure to UV radiation. Suitable sunscreens that may be included in the compositions of the invention are set forth on page 582 of the CTFA Cosmetic Ingredient Handbook, Second Edition, 1992, as well as U.S. Pat. No. 5,620,965, both of which are hereby incorpated by reference. Further examples of chemical and physical sunscreens include those set forth below.

1. UVA Chemical Sunscreens

The term “UVA sunscreen” means a chemical compound that blocks UV radiation in the wavelength range of about 320 to 400 nm. Preferred UVA sunscreens are dibenzoylmethane compounds having the general formula: 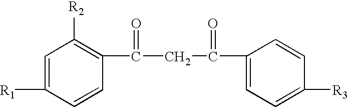
wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isorpoyl-4′-methoxydibenzoymethane, 2-metyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

The claimed compositions may contain from about 0.001-20%, preferably 0.005-5%, more preferably about 0.005-3% by weight of the composition of UVA sunscreen. In one preferred embodiment of the invention the UVA sunscreen is Avobenzone, and it is present at not greater than about 3% by weight of the total composition.

2. UVB Chemical Sunscreens

The term “UVB sunscreen⇄ means a compound that blocks UV radiation in the wavelength range of from about 290 to 320 nm. A variety of UVB chemical sunscreens exist including α-cyano-β,β-diphenyl acrylic acid esters as set forth in U.S. Pat. No. 3,215,724, which is hereby incorporated by reference in its entirety. Particularly preferred is Octocrylene, which is 2-ethylhexyl 2-cyano-3,3-diphenylacrylate. Preferred is where the composition contains no more than about 10% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula: 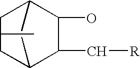
wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula: 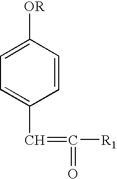
wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at nor more than about 3% by weight of the total composition.

Also suitable as the UVB screening agents are various benzophenone derivatives having the general formula: 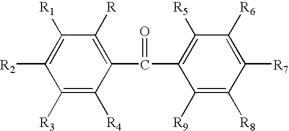
R through R₉ are each independently H, OH, NaO₃S, SO₃H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 1 1, and 12. Particularly preferred is where the benzophenone derivative is Benzophenone 3 (also referred to as Oxybenzone) and Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula: 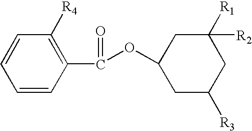
wherein R₁, R₂, R₃, and R₄ are each independently H, OH, NH₂, or C₁₋₂₀ straight or branched chain alkyl. Particularly preferred is where R₁, R₂, and R₃ are methyl and R4 is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula: 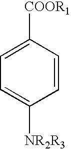
Wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: 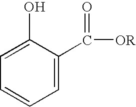
wherein R is a straight or branched chain alkyl, including derivatives of the above compound formed from mono-, di-, or triethanolamines. Particular preferred are octyl salicylate, TEA-salicylcate, DEA-salicylate, and mixtures thereof.

Generally, the amount of the UVB chemical sunscreen present may range from about 0.001-45%, preferably 0.005-40%, more preferably about 0.01-35% by weight of the total composition.

3. Physical Sunscreens

The composition may also contain one or more physical sunscreens. The term “physical sunscreen” means a material that is generally particulate in form that is able to block UV rays by forming an actual physical block on the skin. Examples of particulates that serve as solid physical sunblocks include titanium dioxide, zinc oxide and the like in particle sizes ranging from about 0.001-50 microns, preferably less than 1 micron.

H. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

I. Humectants

If desired, the compositions of the invention comprise about 0.01-30%, preferably about 0.5-25%, more preferably about 1-20% by weight of the total composition of one or more humectants. Suitable humectants include di- or polyhydric alcohols such as glycols, sugars, and similar materials. Suitable glycols include alkylene glycols such as propylene, ethylene, or butylene glycol; or polymeric alkylene glycols such as polyethylene and polypropylene glycols, including PEG 4-240, which are polyethylene glycols having from 4 to 240 repeating ethylene oxide units. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on.

J. Other Botanical Extracts

It may be desirable to include one or more additional botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including acacia (dealbata, farnesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2.

K. Water Soluble Gellants

If the composition is in the emulsion form, it may be desireable to include other water soluble gellants in the water phase of the composition to provide thickening. Such gellants may be included a range of about 0.1-20%, preferably about 1-18%, more preferably about 2-10% by weight of the total composition is suggested, if present. Suitable gellants include soaps, i.e. salts of water insoluble fatty acids with various bases. Examples of soaps include the aluminum, calcium, magnesium, potassium, sodium, or zinc salts of C₆₋₃₀, preferably C₁₀₋₂₂ fatty acids.

Also suitable are hydrocolloids such as gellan gum, gum arabic, carrageenan, and those set forth in U.S. Pat. No. 6,197,319 which is hereby incorporated by reference in its entirety.

L. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

M. Emulsion Stabilizers

If the composition of the invention is in the emulsion form, it may be desirable to incorporate one or more emulsion stabilizers in the composition. If so, suggested ranges are about 0.0001-5%, preferably about 0.0005-3%, more preferably about 0.001-2% by weight of the total composition. Suitable emulsion stabilizers include salts of alkali or alkaline earth metal chlorides or hydroxides, such as sodium chloride, potassium chloride, and the like.

N: Other Film Forming Polymers

It may be desired for the cosmetic composition to contain one or more additional film forming polymers. Such polymers may be silicones or polymers with repeating organic moieties. If present, such film forming polymers are found in ranges of about 0.001-50%, preferably about 0.01-45%, more preferably about 0.1-20% by weight of the total composition. Such film forming polymers may be present in the form of dispersed or solvated particles in water, or in other non-aqueous solvents such as paraffinic hydrocarbons, silicone oils, or organic oils. Examples of such film forming polymers include those set forth below.

1. Copolymers of Silicone and Ethylenically Unsaturated Monomers

One type of film forming polymer that may be used in the compositions of the invention is obtained by reacting silicone moieties with ethylenically unsaturated monomers. The resulting copolymers may be graft or block copolymers. The term “graft copolymer” is familiar to one of ordinary skill in polymer science and is used herein to describe the copolymers which result by adding or “grafting” polymeric side chain moieties (i.e. “grafts”) onto another polymeric moiety referred to as the “backbone”. The backbone may have a higher molecular weight than the grafts. Thus, graft copolymers can be described as polymers having pendant polymeric side chains, and which are formed from the “grafting” or incorporation of polymeric side chains onto or into a polymer backbone. The polymer backbone can be a homopolymer or a copolymer. The graft copolymers are derived from a variety of monomer units.

One type of polymer that may be used as the film forming polymer is a vinyl-silicone graft or block copolymer having the formula: 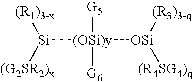
wherein G₅ represents monovalent moieties which can independently be the same or different selected from the group consisting of alkyl, aryl, aralkyl, alkoxy, alkylamino, fluoroalkyl, hydrogen, and -ZSA; A represents a vinyl polymeric segment consisting essentially of a polymerized free radically polymerizable monomer, and Z is a divalent linking group such as C₁₋₁₀ alkylene, aralkylene, arylene, and alkoxylalkylene, most preferably Z is methylene or propylene.

G₆ is a monovalent moiety which can independently be the same or different selected from the group consisting of alkyl, aryl, aralkyl, alkoxy, alkylamino, fluoroalkyl, hydrogen, and -ZSA;
G2 comprises A;
G₄ comprises A;
R₁ is a monovalent moiety which can independently be the same or different and is selected from the group consisting of alkyl, aryl, aralkyl, alkoxy, alkylamino, fluoroalkyl, hydrogen, and hydroxyl; but preferably C₁₋₄ alkyl or hydroxyl, and most preferably methyl.
R₂ is independently the same or different and is a divalent linking group such as C₁₋₁₀ alkylene, arylene, aralkylene, and alkoxyalkylene, preferably C₁₋₃ alkylene or C₇₋₁₀ aralkylene, and most preferably —CH₂— or 1,3-propylene, and
R₃ is a monovalent moiety which is independently alkyl, aryl, aralkyl, alkoxy, alkylamino, fluoroalkyl, hydrogen, or hydroxyl, preferably C₁₋₄ alkyl or hydroxyl, most preferably methyl;
R₄ is independently the same or different and is a divalent linking group such as C₁₋₁₀ alkylene, arylene, aralkylene, alkoxyalkylene, but preferably C₁₋₃ alkylene and C₇₋₁₀ alkarylene, most preferably —CH₂— or 1,3-propylene.
x is an integer of 0-3;
y is an integer of 5 or greater; preferably 10 to 270, and more preferably 40-270; and
q is an integer of 0-3.

These polymers are described in U.S. Pat. No. 5,468,477, which is hereby incorporated by reference. Most preferred is poly(dimethylsiloxane)-g-poly(isobutyl methacrylate), which is manufactured by 3-M Company under the tradename VS 70 IBM. This polymer may be purchased in the dry particulate form, or as a solution where the polymer is dissolved or dispersed in one or more of the liquids that may be found in the composition such as volatile oils (isododecane), water, or other non-volatile or volatile oils. Preferred is where the polymer is in dry particulate form, and as such it can be dissolved in one or more of the liquids comprising the liquid carrier. This polymer has the CTFA name Polysilicone-6.

Another type of such a polymer comprises a vinyl, methacrylic, or acrylic backbone with pendant siloxane groups and pendant fluorochemical groups. Such polymers preferably comprise comprise repeating A, C, D and optionally B monomers wherein:
A is at least one free radically polymerizable acrylic or methacrylic ester of a 1,1,-dihydroperfluoroalkanol or analog thereof, omega-hydridofluoroalkanols, fluoroalkylsulfonamido alcohols, cyclic fluoroalkyl alcohols, and fluoroether alcohols,
B is at least one reinforcing monomer copolymerizable with A,
C is a monomer having the general formula X(Y)nSi(R)3-m Z.m wherein
X is a vinyl group copolymerizable with the A and B monomers,
Y is a divalent linking group which is alkylene, arylene, alkarylene, and aralkylene of 1 to 30 carbon atoms which may incorporate ester, amide, urethane, or urea groups,
n is zero or 1;
m is an integer of from 1 to 3,
R is hydrogen, C₁₋₄ alkyl, aryl, or alkoxy,
Z is a monovalent siloxane polymeric moiety; and
D is at least one free radically polymerizable acrylate or methacrylate copolymer.

Such polymers and their manufacture are disclosed in U.S. Pat. Nos. 5,209,924 and 4,972,037, which are hereby incorporated by reference. More specifically, the preferred polymer is a combination of A, C, and D monomers wherein A is a polymerizable acrylic or methacrylic ester of a fluoroalkylsulfonamido alcohol, and where D is a methacrylic acid ester of a C₁₋₂ straight or branched chain alcohol, and C is as defined above. Most preferred is a polymer having moieties of the general formula: 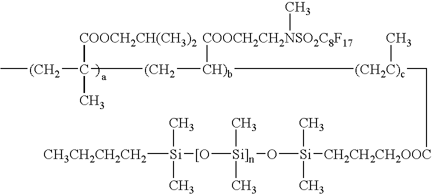
wherein each of a, b, c, and n have a value in the range of 1-100,000, and the terminal groups are selected from the group consisting of a C₁₋₂₀ straight or branched chain alkyl, aryl, and alkoxy and the like. These polymers may be purchased from Minnesota Mining and Manufacturing Company under the tradenames “Silicone Plus” polymers. Most preferred is poly(isobutyl methacrylate-co-methyl FOSEA)-g-poly(dimethylsiloxane) which is sold under the tradename SA 70-5 IBMMF or Polysilicone 7.

Another suitable silicone acrylate copolymer is a polymer having a vinyl, methacrylic, or acrylic polymeric backbone with pendant siloxane groups. Such polymers as disclosed in U.S. Pat. Nos. 4,693,935, 4,981,903, 4,981,902, and which are hereby incorporated by reference. Preferably, these polymers are comprised of A, C, and optionally B monomers wherein:
A is at least on free radically polymerizable vinyl, methacrylate, or acrylate monomer;
B, when present, is at least one reinforcing monomer copolymerizable with A,
C is a monomer having the general formula:
   X(Y)ₙSi(R)₃₋ₘZₘ
   wherein:
X is a vinyl group copolymerizable with the A and B monomers;
Y is a divalent linking group;
n is zero or 1;
m is an integer of from 1 to 3;
R is hydrogen, C₁₋₁₀ alkyl, substituted or unsubstituted phenyl, C₁₋₁₀ alkoxy; and
Z is a monovalent siloxane polymeric moiety.

Examples of A monomers are lower to intermediate methacrylic acid esters of C₁₋₁₂ straight or branched chain alcohols, styrene, vinyl esters, vinyl chloride, vinylidene chloride, acryloyl monomers, and so on.

The B monomer, if present, is a polar acrylic or methacrylic monomer having at least one hydroxyl, amino, or ionic group (such as quaternary ammonium, carboxylate salt, sulfonic acid salt, and so on).

The C monomer is as above defined.

Examples of other suitable copolymers that may be used herein, and their method of manufacture, are described in detail in U.S. Pat. No. 4,693,935, Mazurek, U.S. Pat. No. 4,728,571, and Clemens et al., both of which are incorporated herein by reference. Additional grafted polymers are also disclosed in EPO Application 90307528.1, published as EPO Application 0 408 311, U.S. Pat. No. 5,061,481, Suzuki et al., U.S. Pat. No. 5,106,609, Bolich et al., U.S. Pat. No. 5,100,658, Bolich et al., U.S. Pat. No. 5,100,657, Ansher-Jackson, et al., U.S. Pat. No. 5,104,646, Bolich et al., U.S. Pat. No. 5,618,524, issued Apr. 8, 1997, all of which are incorporated by reference herein in their entirety.

2. Polymers from Ethylenically Unsaturated Monomers

Also suitable for use as film forming polymers are polymers made by polymerizing one or more ethylenically unsaturated monomers. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on.

Preferred are polymers containing one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 2 to 20, more preferably 2 to 8 carbon atoms. If desired, the aliphatic alcohol may have one or more hydroxy groups. Also suitable are methacrylic acid or acrylic acid esters esterified with moieties containing alicyclic or bicyclic rings such as cyclohexyl or isobomyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers include those of the formula: 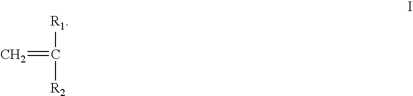
wherein R₁ is H, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or COOM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl groups, or [(CH₂)ₘO]nH wherein m is 1-20, and n is 1-200.

Preferably, the monofunctional ethylenically unsaturated monomer is of Formula I, above, wherein R₁ is H or a C₁₋₃₀ alkyl, and R₂ is COOM wherein M is a C₁₋₃₀ straight or branched chain alkyl which may be substituted with one or more hydroxy groups.

More preferably, R₁ is H or CH₃, and R₂ is COOM wherein M is a C₁₋₁₀ straight or branched chain alkyl which may be substituted with one or more hydroxy groups. In the preferred embodiment of the invention, the monofunctional ethylenically unsaturated monomer is a mixture of monomers of Formula I where in one monomer R₁ is H or CH₃ and R₂ is COOM where M is a C₁₋₁₀ alkyl, and where in the second monomer R₁ is H or CH₃, and R₂ is COOM where M is a C₁₋₁₀ alkyl substituted with one or more hydroxy groups.

Di-, tri- and polyfunctional monomers, as well as oligomers, of the above monofunctional monomers may also be used to form the polymer. Suitable difunctional monomers include those having the general formula: 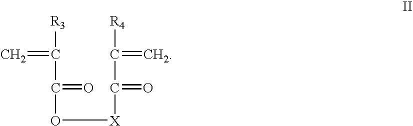
wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100. Particularly preferred are difunctional acrylates and methacrylates, such as the compound of formula II above wherein R₃ and R₄ are CH₃ and X is [(CH₂)ₓO_{y}]_{z} wherein x is 1-4; and y is 1-6; and z is 1-10.

Trifunctional and polyfunctional monomers are also suitable for use in the polymerizable monomer to form the polymer used in the compositions of the invention. Examples of such monomers include acrylates and methacrylates such as trimethylolpropane trimethacrylate or trimethylolpropane triacrylate.

The polymers can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622,372; 4,656,233; 4,711,942; 4,681,918; and 4,822,859; all of which are hereby incorporated by reference.

Also suitable are polymers formed from the monomer of Formula I, above, which are cyclized, in particular, cycloalkylacrylate polymers or copolymers having the following general formulas: 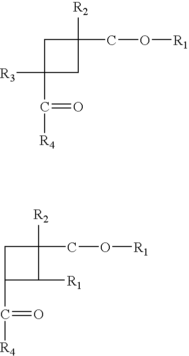
wherein R₁, R₂, R₃, and R₄ are as defined above. Typically such polymers are referred to as cycloalkylacrylate polymers. Such polymers are sold by Phoenix Chemical, Inc. under the tradename Giovarez AC-5099M. Giovarez has the chemical name isododecane acrylates copolymer and the polymer is solubilized in isododecane. The monomers mentioned herein can be polymerized with various types of organic groups such as propylene glycol, isocyanates, amides, etc.

One type of organic group that can be polymerized with the above monomers includes a urethane monomer. Urethanes are generally formed by the reaction of polyhydroxy compounds with diisocyanates, as follows: 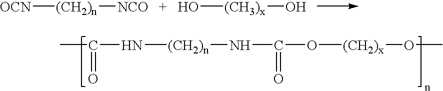
wherein x is 1-1000.

Another type of monomer that may be polymerized with the above comprise amide groups, preferably having the the following formula: 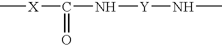
wherein X and Y are each independently linear or branched alkylene having ₁₋₄₀ carbon atoms, which may be substituted with one or more amide, hydrogen, alkyl, aryl, or halogen substituents.

Another type of organic monomer may be alpha or beta pinenes, or terpenes, abietic acid, and the like.

Suitable silicone polymers include silicone esters set forth in U.S. Pat. No. 5,725,845 which is hereby incorporated by reference in its entirety. Other polymers that can enhance adhesion to skin include silicone esters comprising units of the general formula RₐR^{E}_{b}SiO_{[4-(a+b)/2]} or R¹³ₓR^{E}_{y}SiO_{1/2} wherein R and R¹³ are each independently an organic radical such as alkyl, cycloalkyl, or aryl, or, for example, methyl, ethyl, propyl, hexyl, octyl, decyl, aryl, cyclohexyl, and the like, a is a number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Preferred R_{E} radicals are those wherein the ester group is formed of one or more fatty acid moieities (e.g. of about 2, often about 3 to 10 carbon atoms) and one or more aliphatic alcohol moieities (e.g. of about 10 to 30 carbon atoms). Examples of such acid moieities include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxy)propane. Preferably the ester subgroup (i.e. the carbonyloxy radical) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety. Silicone esters having the above formula are disclosed in U.S. Pat. No. 4,725,658 and U.S. Pat. No. 5,334,737, which are hereby incorporated by reference. Preferred silicone esters are the liquid siloxy silicates disclosed in U.S. Pat. No. 5,334,737, e.g. diisostearoyl trimethylolpropane siloxysilicate (prepared in Examples 9 and 14 of this patent), and dilauroyl trimethylolpropane siloxy silicate (prepared in Example 5 of 'the patent), which are commercially available from General Electric under the tradenames SF 1318 and SF 1312, respectively.

Silicone gums or other types of silicone solids may be also used in the compositions of the invention. Examples of silicone gums include those set forth in U.S. Pat. No.6,139,823, which is hereby incorporated by reference. Preferred gums have a viscosity ranging from 600,000 to 1,000,000 centipoise at 25° C.

Also suitable are silicone resins, also referred to as MQ or MT resins. Such silicones have the general formula:
[R₃SiO_{0.5}]ₓ[SiO_{2.0}]_{y}
or
[R₃SiO_{0.5}]ₓ[SiO_{1.5}]_{y}
wherein R is a C1-6 straight or branched chain alkyl, preferably methyl; and x and y are each independently 1-1,000.

Such silicone resins are referred to as trimethylsiloxysilicate, or methylpolysilsesquioxane.

3. Natural Polymers

Also suitable for use are one or more naturally occuring polymeric materials such as resinous plant extracts including such as rosin, shellac, and the like.

III. Forms of the Cosmetic Composition

A. Foundation Makeup, Color Cosmetics

The cosmetic compositions may be in the form of foundation makeup or color cosmetics such as eyeshadow, blush, concealer, eyeliner, mascara, lip gloss, brow color; and in the liquid, cream, solid, or stick form. Suitable foundation makeup compositions may be water-in-oil or oil-in-water emulsions. Such compositions generally comprise about:
0.001-80% of the IPN,
0.5-95% water,
0.5-25% particulate matter,
0.01-20% surfactant, and
0.1-95% nonvolatile or volatile oil.

In addition, these composition may further contain ingredients selected from the group of humectants, preservatives, gellants, and all of the ingredients as set forth above in the ranges set forth herein.

Various anhydrous color cosmetic products may also be suitable, such as blush, powder, lipsticks, lip gloss, eyeshadows, and the like. Such anhydrous color cosmetic compositions may generally comprise about:
0.001-80% of the IPN,
0.1-99% oil; and optionally
0.1-80% particulate matter; and optionally
0.001-50% thickening agent or wax.

The compositions may additionally contain the various other ingredients set forth above and in the ranges taught.

Preferably, the compositions are in the form of a lipcolor or lipstick, which may be a composition for coloring the lips that is in liquid, semi-solid, or solid form.

Alternatively, the composition may be in the form of a base lip color, which is a lip color applied to the lips as a basecoat to provide color, followed by application of a separate gloss coat which comprises one or more oils or waxes that provide shine, moisturization, or similar benefits to the layers applied to the lips. Examples of such lip compositions and topcoats are disclosed in U.S. patent application No. 2002/0159960, entitled “Method for Improving the Properties of Transfer Resistant Lip Compositions and Related Compositions and Articles”, claiming priority from provisional application No. 60/271,849, filed Feb. 27, 2001; which is hereby incorporated by reference in its entirety.

B. Lotions, Creams Gels, and Sunscreens

The cosmetic compositions of the invention may be in the form of lotions, gels or sunscreens. Suitable skin care lotions and creams are in the emulsion form, and may be water-in-oil or oil-in-water emulsions, preferably oil-in-water emulsions. Creams, lotions, and/or may contain the following ranges of ingredients:
about 0.001-80% of the IPN,
about 0.1-90% oil,
about 0.01-20% surfactant; and
about 0.001-95% water.

C. Skin and Hair Cleansing and Conditioning Compositions

Skin and hair cleansing and conditioning compositions such as facial cleansers, shampoos, hair conditioners and the like are also suitable cosmetic compositions in accordance with the invention.

Generally skin and hair cleansing compositions comprise about:
0.001-80% of the IPN,
1-95% water, and
0.1-40% surfactant, preferably an anionic, amphoteric, or zwitterionic surfactant.
0.01-40% oil.

Suitable hair conditioner compositions comprise:
0.001-80% of the IPN,
0.1-20% cationic surfactant,
0.1-30% fatty alcohol,
0.001-10% nonionic surfactant, and
5-95% water.

Suitable cationic and nonionic surfactants are as mentioned herein. Examples of suitable fatty alcohols include those having the general formula R—OH, wherein R is a C₆₋₃₀ straight or branched chain, saturated or unsaturated alkyl.

D. Nail Enamel Compositions

The cosmetic compositions of the invention may also be in the form of nail enamel compositions. Such compositions generally comprise:
0.001-80% of the IPN,
0.01-80% solvent,
0.001-40% particulate matter, and
optionally 0.01-40% of one or more polymers such as cellulosic polymers, acrylate polymers, and the like.

Suitable solvents include acetone, alkyl acetates such as ethyl acetate butyl acetate and the like, alkyl ethers such as propylene glycol monomethyl ether, and the like.

III. The Method

The invention is directed to improving the transfer resistance or long wearing property of a cosmetic composition by formulating the composition with an IPN. The IPN will cause the cosmetic composition to exhibit an improved transfer resistant or long wearing finish when applied to skin, lips, or hair. The term “improved transfer resistance” means that the finish will exhibit an improved ability to resist transfer from the keratinous surface to which it is applied when such surface is touched to another surface such as skin, tissue, or utensils. The term “improved long wear property” means that the composition will exhibit improved wear, and possibly, adhesion, to the surface to which it is applied.

The invention is further directed to a method for reducing the shiny appearance of skin or otherwise improving the appearance of skin imperfections comprising applying to the skin a cosmetic composition comprising at least one IPN.

The invention will be further described in connection with the following examples which are set forth for the purposes of illustration only.

### EXAMPLE 1

A lipstick composition according to the invention was made as follows:

| | w/w % |
|---|---|
| Candelilla wax | 1.50 |
| Carnauba wax | 1.25 |
| Synthetic wax | 2.08 |
| Ozokerite | 5.00 |
| Cetyl alcohol | 1.67 |
| Ethylhexyl palmitate | 10.00 |
| Isostearyl hydroxystearate | 8.33 |
| Caprylic/capric triglyceride | 3.54 |
| Octyldodecanol | 5.00 |
| Castor oil | 25.10 |
| Tocopheryl acetate | 0.08 |
| Mineral Oil, aloe barbadensis leaf extract | 0.08 |
| Retinyl palmitate | 0.08 |
| Ascorbyl palmitate | 0.08 |
| Methylparaben | 0.25 |
| Propylparaben | 0.08 |
| BHT | 0.08 |
| Sorbic acid | 0.17 |
| Titanium dioxide/castor oil | 0.70 |
| Red 6 Lake/castor oil | 0.64 |
| Red 7 Lake/castor oil | 0.64 |
| Yellow 5 Lake/castor oil | 0.90 |
| Black iron oxide/castor oil | 0.17 |
| Red iron oxide/castor oil | 0.33 |
| Mica, titanium dioxide | 2.25 |
| Mica, titanium dioxide, iron oxides | 0.75 |
| Mica | 7.59 |
| Mica, silica | 1.67 |
| Silica | 1.67 |
| Gransil EPSQ* | 5.00 |
| Castor oil | QS |

*Interpenetrating Polymer Network, polydimethylsiloxane and polymethylsilsesquioxane.

The composition was prepared by combining the pigments and powders and grinding in a roller mill with a portion of the oil. The waxes were melting and combined with the oils. The pigment grind was added, the mixture stirred well, and poured into lipstick molds and allowed to harden.

### EXAMPLE 2

An anhydrous cosmetic formula suitable for use as eye shadow or blush was made as follows:

| | w/w % |
|---|---|
| Cyclomethicone (D5/D6) | 19.59 |
| Neopentyl glycol diethylhexanoate | 17.68 |
| Isotridecyl isononanoate | 12.52 |
| Dimethicone (6 centistokes) | 8.98 |
| Trimethylsiloxysilicate | 17.14 |
| Iron oxides/methicone | 0.61 |
| Methyl paraben | 0.27 |
| Propyl paraben | 0.14 |
| Ethyl paraben | 0.14 |
| Butyl paraben | 0.41 |
| Trisodium EDTA | 0.40 |
| Mica, methicone | 1.39 |
| Lauroyl lysine | 2.00 |
| Bismuth oxychloride | 0.95 |
| Silica | 0.68 |
| Acrylates-copolymer | 0.54 |
| Polyethylene | 0.20 |
| Alumina | 3.95 |
| Dimethicone/cyclomethicone | 1.90 |
| Polyisobutene | 0.48 |
| Polyglycery-4-isostearate, cetyl PEG/PPG | 4.08 |
| 10/1 dimethicone, hexyl laurate | |
| Tribehenin | 0.27 |
| Mica, titanium dioxide | 2.04 |
| Mica, titanium dioxide, iron oxides | 10.2 |
| Gransil EPSQ* | 11.16 |
| Isododecane | QS |

*Interpenetrating Polymer Network, polydimethylsiloxane and polymethylsilsesquioxane.

The composition was prepared by combining the ingredients and mixing well.

### EXAMPLE 3

A water based color cosmetic composition suitable for use as an eye shadow or blush was made as follows:

| | w/w % |
|---|---|
| Water | QS |
| Trisodium EDTA | 0.99 |
| Butylene glycol | 1.89 |
| Magnesium aluminum silicate | 0.33 |
| Cellulose gum | 0.10 |
| Methyl paraben | 0.33 |
| Ethyl paraben | 0.05 |
| Lecithin, polysorbate 20, sorbitan laurate, propylene | 0.24 |
| glycol stearate, propylene glycol laurate | |
| Iron oxides | 0.07 |
| Silica | 0.94 |
| Boron nitride | 1.89 |
| Butylene glycol | 0.94 |
| Xanthan gum | 0.14 |
| Mica | 0.68 |
| Mica, titanium dioxide | 13.12 |
| Mica, iron oxides, titanium dioxide | 1.51 |
| Isotridecyl isononanoate | 9.44 |
| C10-30 cholesterol/lanosterol esters | 0.47 |
| Cetyl alcohol | 2.36 |
| Steareth-20 | 1.32 |
| Sorbitan stearate | 0.94 |
| Choleth-24, ceteth-24 | 0.14 |
| Glyceryl stearate, PEG-100 stearate | 0.09 |
| Cyclohexasiloxane, cyclopentasiloxane | 3.78 |
| Propyl paraben | 0.09 |
| Phenoxyethanol | 0.94 |
| PVP | 0.28 |
| Mica | 0.01 |
| Gransil EPSQ* | 5.60 |

*Interpenetrating Polymer Network, polydimethylsiloxane and polymethylsilsesquioxane.

The composition was prepared by combining the ingredients and mixing well.

### EXAMPLE 4

A facial or body lotion was made as follows:

| | w/w % |
|---|---|
| Water | QS |
| Carbopol | 0.3 |
| Magnesium ascorbyl phosphate | 0.01 |
| Butylene glycol | 5.00 |
| Methyl paraben | 0.25 |
| Ethyl paraben | 0.15 |
| Glycerin | 5.00 |
| Dimethicone | 0.50 |
| Caprylic/capric triglyceride | 10.00 |
| C12-15 alkyl benzoate | 5.00 |
| SilDerm Powder EPSQ | 2.50 |
| Cetyl alcohol | 0.50 |
| Stearyl alcohol | 0.75 |
| Polysorbate 60 | 1.00 |
| Stearic acid | 3.20 |
| Propyl paraben | 0.10 |
| Sorbitan stearate | 1.00 |
| Paraffin | 1.00 |
| Tocopheryl acetate | 0.10 |
| Retinyl palmitate | 0.001 |
| Trisodium EDTA | 0.10 |
| Triethanolamine | 1.50 |
| Diazolidinyl urea | 0.20 |

The water was heated to 80 to 85° C. The carbopol was sifted into the water. When completely wet out, the mixture was mixed with a T blade. The magnesium ascorbyl phosphate was added to a separate beaker, and the mixture of butylene glycol and parabens to another separate beaker. The two separate beakers were added to the mixture of water and carbopol along with the remaining ingredients.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

1. A cosmetic composition comprising an interpenetrating polymer network of interlaced, noncovalently bonded, polydimethylsiloxane and polymethylsilsesquioxane in a cosmetically acceptable carrier.

2. The composition of claim 1 which is an anhydrous color cosmetic product.

3. The composition of claim 2, which is an eyeshadow, blush, lipstick, lip gloss, nail enamel, or powder.

4. The composition of claim 3 wherein the oil comprises at least one volatile oil.

5. The composition of claim 4 wherein the volatile oil is present ranging from about 0. 1-80% by weight of the total composition and comprises a linear or cyclic silicone oil, or a paraffinic hydrocarbon.

6. The composition of claim 5 wherein the volatile oil is selected from the group consisting of: (a) a linear silicone selected from the group consisting of hexamethyldisiloxane, octamethyltrisiloxane, decamethyltetrasiloxane, dodecamethylpentasiloxane, and mixtures thereof, (b) a cyclic silicone selected from the group consisting of octamethylcyclotetrasiloxane, decamethylcyclopentasiloxane, dodecamethylcyclohexasiloxane, and mixtures thereof, (c) a paraffinic hydrocarbon is selected from the group consisting of isododecane, isohexadecane; and mixtures thereof.

7. The composition of claim 6 wherein the volatile oil comprises a linear volatile silicone selected from the group consisting of octamethyltrisiloxane, decamethyltetrasiloxane, dodecamethylpentasiloxane, and mixtures thereof.

8. The composition of claim 6 wherein the volatile oil comprises a cyclic silicone selected from the group consisting of decamethylcyclopentasiloxane, dodecamethylcyclohexasiloxane, and mixtures thereof.

9. The composition of claim 6 wherein the volatile oil comprises a paraffinic hydrocarbon selected from the group consisting of isododecane, isohexadecane, and mixtures thereof.

10. The composition of claim 6 further comprising at least one non-volatile oil.

11. The composition of claim 6 wherein the non-volatile oil is present ranging from about 0.1-70% by weight of the total composition and comprises a silicone oil, hydrocarbon, ester, fluorinated oil, or mixtures thereof.

12. The composition of claim 11 wherein the silicone oil comprises dimethicone, dimethicone copolyol, phenyl trimethicone, or mixtures thereof.

13. The composition of claim 11 wherein the ester comprises a monoester, diester, triester, or mixtures thereof.

14. The composition of claim 11 further comprising particulates.

15. The composition of claim 14 wherein the particulates are present ranging from about 0.001-95% by weight of the total composition and are pigments, powders, or mixtures thereof.

16. The composition of claim 11 further comprising at least one wax.

17. The composition of claim 11 wherein the at least one wax is present ranging from about 0.1-50% by weight of the total composition, and is an animal, vegetable, mineral, or silicone wax, or mixtures thereof.

18. The composition of claim 17 wherein the wax comprises a homo- or copolymer of ethylene.

19. The composition of 11 further comprising at least one Theological additive.

20. The composition of claim 19 wherein the rheological additive is present ranging from about 0.01-60% by weight of the total composition, and comprises montmorillonite minerals, associative thickeners, silicone elastomers, or mixtures thereof.

21. The composition of claim 1 which is an anhydrous transfer resistant or long wearing lipstick composition comprising:
a volatile oil selected from the group consisting of octamethyltrisiloxane, decamethyltetrasiloxane, dodecamethylpentasiloxane, octamethylcyclotetrasiloxane, decamethylcyclopentasiloxane, dodecamethylhexasiloxane, isododecane, isohexadecane, and mixtures thereof,
a non-volatile oil selected from the group consisting of silicone oil, ester, hydrocarbon, fluorinated oil, and mixtures thereof,
particulates selected from the group consisting of pigments, powders, and mixtures thereof,
a wax selected from the group consisting of animal wax, vegetable wax, mineral wax, silicone wax, and mixtures thereof.

22. The composition of claim 21 wherein the non-volatile oil is present ranging from 0.1-70% by weight of the total composition and comprises dimethicone, dimethicone copolyol, phenyl trimethicone, and mixtures thereof.

23. The composition of claim 21 wherein the non-volatile oil comprises phenyl trimethicone.

24. The composition of claim 21 wherein the non-volatile oil comprises dimethicone.

25. The composition of claim 21 wherein the wax comprises a homo- or copolymer of ethylene.

26. The composition of claim 1 which is an eyeshadow or blush comprising:
a volatile oil selected from the group consisting of octamethyltrisiloxane, decamethyltetrasiloxane, dodecamethylpentasiloxane, octamethylcyclotetrasiloxane, decamethylcyclopentasiloxane, dodecamethylhexasiloxane, isododecane, isohexadecane, and mixtures thereof,
a non-volatile oil selected from the group consisting of silicone oil, ester, hydrocarbon, fluorinated oil, and mixtures thereof,
particulates selected from the group consisting of pigments, powders, and mixtures thereof,
a wax selected from the group consisting of animal wax, vegetable wax, mineral wax, silicone wax, and mixtures thereof.

27. The composition of claim 26 wherein the volatile oil is selected from the group consisting of octamethylcyclotetrasiloxane, decamethylcyclopentasiloxane, dodecamethylhexasiloxane, and mixtures thereof.

28. The composition of claim 26 wherein the non-volatile oil comprises dimethicone, phenyl trimethicone, dimethicone copolyol, cetyl dimethicone copolyol, and mixtures thereof.

29. The composition of claim 26 wherein the wax comprises a homo- or copolymer of ethylene.

30. The composition of claim 1 which is a lipstick comprising:
a non-volatile oil,
wax, and
particulates.

31. The composition of claim 30 wherein the non-volatile oil comprises an ester, a silicone, or mixtures thereof.

32. The composition of claim 30 wherein the wax comprises a homo- or copolymer of ethylene.

33. The composition of claim 30 wherein the particulates are pigments, powders, or mixtures thereof.

34. The composition of claim 1 wherein the cosmetically acceptable carrier is a water and oil emulsion comprising from about 0.1-95% water and from about 0.1-95% oil.

35. The composition of claim 34 wherein the oil comprises at least one volatile oil selected from the group consisting of octamethyltrisiloxane, decamethyltetrasiloxane, dodecamethylpentasiloxane, octamethylcyclotetrasiloxane, decamethylcyclopentasiloxane, dodecamethylhexasiloxane, isododecane, isohexadecane, and mixtures thereof,

36. The composition of claim 35 comprising at least one non-volatile oil.

37. The composition of claim 36 wherein the non-volatile oil comprises a silicone oil, an ester, a fluorinated oil, or mixtures thereof.

38. The composition of claim 36 further comprising at least one surfactant.

39. The composition of claim 38 wherein the surfactant is present ranging from about 0.01-20% by weight of the total composition and comprises a nonionic surfactant which is an alkoxylated alcohol, alkoxylated carboxylic acid, sorbitan, or mixtures thereof.

40. The composition of claim 36 further comprising one or more sunscreens.

41. The composition of claim 36 further comprising one or more humectants.

42. The composition of claim 36 further comprising at least one film forming polymer.

43. The composition of claim 42 wherein the film forming polymer is present ranging from about 0.01-45% by weight of the total composition and are copolymers of silicone and ethylenically unsaturated monomers, polymers from ethylenically unsaturated monomers, silicone polymers, and mixtures thereof.

44. The composition of claim 36 which is a skin or body lotion.

45. A method for increasing the transfer resistance or long wearing property of a cosmetic composition comprising formulating said composition with at least one interpenetrating polymer network in a cosmetically acceptable carrier.

46. The method of claim 45 wherein the interpenetrating polymer network is present ranging from about 0.001-85% by weight of the total composition.

47. The method of claim 46 wherein composition comprises an interpenetrating polymer network of interlaced, noncovalently bonded, polydimethylsiloxane and polymethylsilsesquioxane in a cosmetically acceptable carrier.

48. A method for reducing the shiny appearance of skin or otherwise improving skin imperfections comprising applying to the skin a cosmetic composition comprising at least one interpenetrating polymer network in a cosmetically acceptable carrier.

49. The method of claim 48 wherein the interpenetrating polymer network is present ranging from about 0.001-85% by weight of the total composition.

50. The method of claim 49 wherein the composition comprises an interpenetrating polymer network of interlaced, noncovalently bonded, polydimethylsiloxane and polymethylsilsesquioxane in a cosmetically acceptable carrier.
